(19) United States
(12) Patent Application Publication
(10) Pub. No.: US 2020/0054659 A1
(43) Pub. Date: Feb. 20, 2020

(54) EXTENDED RELEASE CAPECITABINE CAPSULES

(71) Applicant: Intas Pharmaceuticals Ltd., Ahmedabad, Gujaral (IN)

(72) Inventors: Ashish SEHGAL, Ahmedabad (IN); Manishkumar CHAUHAN, Ahmedabad (IN); Manishkumar PATEL, Ahmedabad (IN); Kavan PANDYA, Ahmedabad (IN); Rushabh BHIMANI, Ahmedabad (IN)

(21) Appl. No.: 15/735,820

(22) PCT Filed: Jun. 13, 2016
(86) PCT No.: PCT/IB2016/053465

§ 371 Date: Dec. 12, 2017

(30) Foreign Application Priority Data
Jun. 13, 2015 (IN) 2280/MUM/2015

(51) Int. Cl. A61K 31/7068

(52) U.S. Cl. CPC A61K 31/7068

(57) ABSTRACT

The present invention relates to an extended release capsules comprising multiple units of Capecitabine, wherein the dissolution of Capecitabine from the said composition is extended up to 12 hours. Further the present invention discloses process for the preparation of the said composition.

### RELATED APPLICATIONS

This application is related to Indian Provisional Application 2280/MUM/2015 filed 13 Jun. 2015 and is incorporated herein in its entirety.

### FILED OF THE INVENTION

The present invention relates to extended release capsules comprising multiple units of Capecitabine, wherein the dissolution of Capecitabine from the said composition is extended up to 12 hours. Further the present invention discloses process for the preparation of the said composition.

### BACKGROUND OF THE INVENTION

Capecitabine is a fluoropyrimidine carbamate having antineoplastic activity. It is an orally administered systemic prodrug of 5′-deoxy-5-fluorouridine (5′-DFUR) which is converted to fluorouracil. Molecular formula of Capecitabine is C₁₅H₂₂FN₃O₆ and the molecular weight is 359.35 and has following chemical structure:

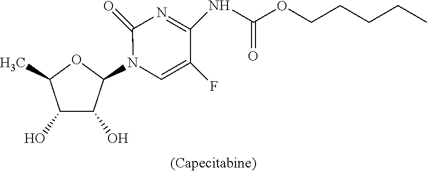 [SMILES: CCCCCOC(=O)Nc1nc(=O)n([C@@H]2O[C@H](C)[C@@H](O)[C@H]2O)cc1F]

U.S. Pat. Nos. 4,966,891 and 5,472,949 discloses Fluorocytidine derivatives and N⁴-(substituted-oxycarbonyl)-5′-deoxy-5-fluorocytidine compounds respectively which cover Capecitabine and methods of using same. Capecitabine is marketed as immediate release tablet comprising 150 or 500 mg Capecitabine for oral administration under trade name XELODA® by Roche. The inactive ingredients in XELODA® include: anhydrous lactose, croscarmellose sodium, hydroxypropyl methylcellulose, microcrystalline cellulose, magnesium stearate and purified water. The peach or light peach film coating contains hydroxypropyl methylcellulose, talc, titanium dioxide, and synthetic yellow and red iron oxides. Capecitabine is indicated for colon cancer, metastatic colorectal cancer and metastatic breast cancer as monotherapy or combination therapy.

Recommended standard starting dose of Capecitabine is 1250 mg/m² administered orally twice daily (morning and evening; equivalent to 2500 mg/m² total daily dose) for 2 weeks followed by a 1-week rest period given as 3-week cycles. In combination with docetaxel, the recommended dose of Capecitabine is 1250 mg/m² twice daily for 2 weeks followed by a 1-week rest period, combined with docetaxel at 75 mg/m² as a 1-hour intravenous infusion every 3 weeks. Capecitabine Tablets should be swallowed with water within 30 minutes after the end of a meal.

Currently available immediate release (IR) composition of Capecitabine has Tₘₐₓ of Approximately 1.5 hours and T_{1/2} of 0.75 hours. Further the available composition does not maintain constant plasma concentration i.e difficult to achieve steady state concentration.

After administration of currently available IR composition of Capecitabine, the plasma concentration of a Capecitabine reaches below minimum effective concentration after approximately 6 hours, which result in no therapeutic effect between 6 to 12 hours after administration of the dose (FIG. 1).

Capecitabine has high therapeutic value for the treatment of cancer. IR tablet of Capecitabine may lead to severe gastrointestinal side effects. Till date, several approaches are reported to overcome the problem associated with IR composition.

WO2013030602 discloses an extended release pharmaceutical composition for oral administration comprising substantially amorphous Capecitabine or an analog thereof and optionally an extended release component, wherein the extended release component is present in an amount of less than 200% w/w, relative to the total weight of the Capecitabine or analog thereof.

WO2006110800 discloses a multiparticulate modified release composition comprising Capecitabine, wherein, following oral delivery, the composition delivers Capecitabine in a pulsatile manner.

US20070122481 discloses a multiparticulate modified release composition comprising a Capecitabine, wherein, following oral delivery, the composition delivers the Capecitabine in a pulsatile manner.

However, still there is need to develop Capecitabine composition which overcomes problems associated with currently available IR tablet, and releases the drug from the composition up to 12 hours after administration, which in-turn shall provide and maintain effective plasma concentration of Capecitabine approximately for 12 hours.

### OBJECT OF THE INVENTION

It is therefore object of the invention is to provide extended release capsules comprising multiple units of Capecitabine, wherein the dissolution of Capecitabine from the said composition is extended up to 12 hours.

Another object of the present invention is to provide process for the preparation of the extended release capsules comprising multiple units of Capecitabine, wherein the dissolution of Capecitabine from the said composition is extended up to 12 hours.

Another object of the present invention is to provide extended release capsules comprising multiple units of Capecitabine, wherein the multiple units are in form of mini tablet.

Another object of the present invention is to provide extended release capsules comprising multiple units of Capecitabine, wherein the multiple units are in form of pellet or sphere.

Another object of the present invention is to provide extended release capsules comprising multiple units of Capecitabine, wherein the multiple units are in form of multi-particulates.

Another object of the present invention is to provide process for the preparation of the extended release capsules comprising multiple units of Capecitabine, wherein the dissolution of Capecitabine from the said composition is 15 to 35% after 1 hour, 40 to 60% after 4 hour, 60 to 80% after 8 hour and not less than (NLT) 85% of the total amount of Capecitabine released after 12 hour.

Another object of the present invention is to provide extended release capsules comprising multiple units of Capecitabine suitable for twice daily dosage regime, wherein dissolution of Capecitabine from the said composition is extended up to 12 hours.

Another object of the present invention is to provide extended release capsules comprising multiple units of Capecitabine which would release the drug from the said composition up to 12 hours after administration.

Another object of the present invention is to provide extended release capsules comprising multiple units of Capecitabine, which shall provide and maintain effective plasma concentration of Capecitabine approximately for 12 hours after administration.

Another object of the present invention is to provide extended release capsules comprising multiple units of Capecitabine wherein the composition comprises Capecitabine ranging from 150 to 750 mg, wherein dissolution of Capecitabine from the said composition is extended up to 12 hours.

Another object of the present invention is to provide extended release capsules comprising multiple units of Capecitabine, wherein the release from the multiple units is controlled by modified release matrix material.

Another object of the present invention is to provide extended release capsules comprising multiple units of Capecitabine, wherein the release from the multiple units is controlled by modified release coating material.

Another object of the present invention is to provide extended release capsules comprising multiple units of Capecitabine, wherein the release from the multiple units is controlled by modified release coating on sphere/pellet comprising Capecitabine.

Another object of the present invention is to provide extended release capsules comprising multiple units of Capecitabine, wherein the release from the multiple units is controlled by modified release coating on multi-particulates comprising Capecitabine.

### SUMMARY OF THE INVENTION

Present invention provides extended release capsules comprising multiple units of Capecitabine, wherein the dissolution of Capecitabine from the said composition is extended up to 12 hours. Further the present invention provides extended release capsules comprising multiple units of Capecitabine, wherein the multiple units are in form of mini tablet or pellet or multi-particulates. Further the present invention provides extended release capsules comprising multiple units of Capecitabine, wherein the release from the multiple units is controlled by modified release matrix material or modified release coating material. Further the present invention provides extended release capsules comprising multiple units of Capecitabine suitable for twice daily dosage regime which releases the drug from the said composition up to 12 hours after administration. Further the present invention provides extended release capsules comprising multiple units of Capecitabine wherein the composition comprises Capecitabine ranging from 150 to 750 mg.

### BRIEF DESCRIPTION OF THE FIGURES

FIG. 1 shows Plasma conc. of Capecitabine Immediate release tablet.

FIG. 2 shows Dissolution profile of Capecitabine extended release capsule 500 mg prepared according to example 2.

FIG. 3 shows Dissolution profile of Capecitabine extended release capsule 500 mg prepared according to example 3.

FIG. 4 shows Dissolution profile of Capecitabine extended release capsule 500 mg prepared according to example 7.

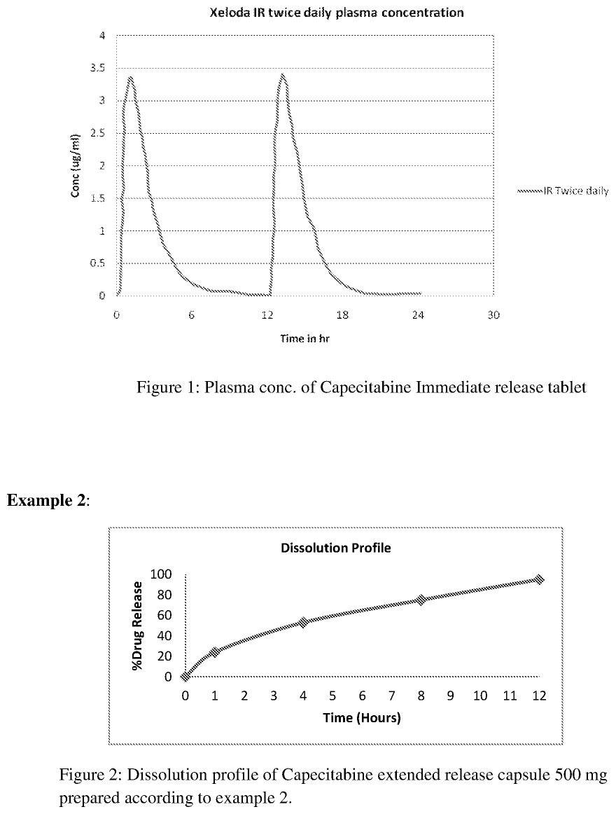

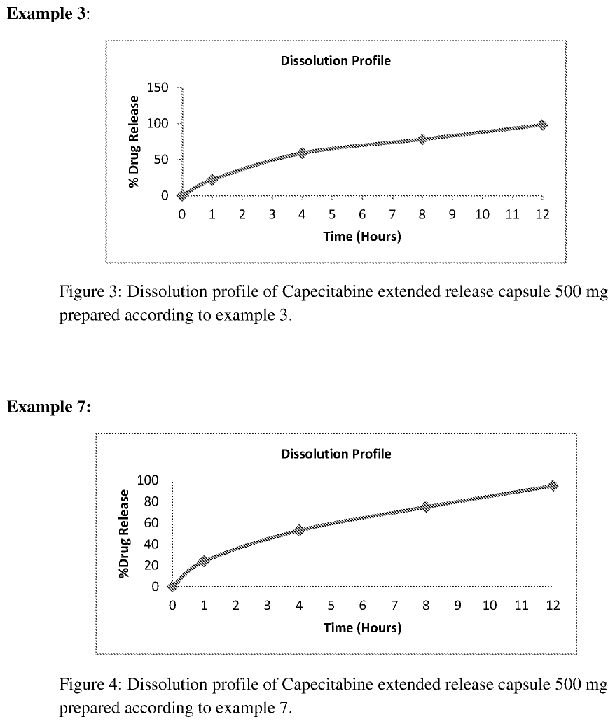

### DETAILED DESCRIPTION OF THE INVENTION

Present invention provides extended release capsules comprising multiple units of Capecitabine, wherein the dissolution of Capecitabine from the said composition is extended up to 12 hours.

In another embodiment, the present invention provides extended release capsules comprising multiple units of Capecitabine suitable for twice daily administration which shall provide and maintain effective plasma concentration of Capecitabine approximately for 12 hours after administration.

In another embodiment, the present invention provides extended release capsules comprising multiple units of Capecitabine, wherein the multiple units are in form of mini tablet or pellet or multi-particulates or bead or granule and like thereof.

In another embodiment, the present invention provides extended release capsules comprising multiple units of Capecitabine, wherein the dissolution of Capecitabine from the said composition is extended up to 12 hours, wherein the multiple units are in form of mini tablet or pellet or multi-particulates or bead or granule and like thereof, wherein the release from the multiple units is controlled by modified release matrix material or modified release coating material and process for preparation thereof.

For the purpose of this specification, the term “composition” means a pharmaceutical composition comprising Capecitabine and pharmaceutically acceptable excipients, wherein the dissolution of the Capecitabine from the said composition is extended up to 12 hours. Further, the said composition is in the form of multiple units, wherein the dissolution of Capecitabine form the said composition is controlled by modified release matrix material or modified release coating material.

For the purpose of this specification, the term “extended release” means a release of drug for a longer duration of time i.e. not immediate release.

For the purpose of this specification, the term “multiple units” means a suitable dosage form which can be incorporated into capsule, for e.g., mini-tablets, pellet/sphere, multi-particulates, bead, granule and like thereof. Further, the multiple units are incorporated into capsule in an amount of more than one unit.

For the purpose of this specification, the term “multi-particulates” means a plurality of spheres, wherein the spheres comprises of coating of Capecitabine layer on an inert core.

According to present invention extended release capsules comprising multiple units of Capecitabine, wherein the release from the multiple units is controlled by modified release matrix material or modified release coating material which further comprises suitable excipients.

According to present invention suitable excipients may include, but not limited to binder, diluent, lubricant, glidant, and like thereof.

According to present invention, binder may include, but not limited to acacia, carbomer, carboxymethylcellulose, cellulose microcrystalline, copovidone, gelatin, guar gum, hydroxypropyl cellulose, low-substituted hydroxypropyl cellulose, hydroxypropyl methylcellulose, hydroxypropyl methylcellulose acetate succinate, methyl cellulose, ethyl cellulose, polyethylene oxide, povidone, starch, pregelatinized starch, ammonio methacrylate copolymer and the like, or mixtures thereof.

According to present invention, diluent may include, but not limited to lactose anhydrous, lactose monohydrate, spray dried lactose, dicalcium phosphate, calcium phosphate tribasic, calcium carbonate, calcium sulfate, starch, corn starch, potato starch, wheat starch, pregelatinized starch, microcrystalline cellulose, silicified microcrystalline cellulose, cellulose microcrystalline powdered and the like, or mixtures thereof.

According to present invention, lubricant may include, but not limited to calcium stearate, glyceryl behenate, magnesium stearate, mineral oil light, polyethylene glycol, castor oil, sodium stearyl fumarate, starch, stearic acid, talc, hydrogenated vegetable oil, zinc stearate, sodium benzoate and the like, or mixtures thereof.

According to present invention, glidant may include, but not limited to calcium silicate, magnesium silicate, colloidal silicon dioxide, talc and the like, or mixtures thereof.

In another embodiment, the present invention provides extended release capsules comprising multiple units of Capecitabine, wherein dissolution of Capecitabine from the said composition is extended up to 12 hours, preferably for at least 10 hours.

In another embodiment, the present invention provides extended release capsules comprising multiple units of Capecitabine, wherein the dissolution of Capecitabine from the said composition is 15 to 35% after 1 hour, 40 to 60% after 4 hour, 60 to 80% after 8 hour and NLT 85% of the total amount of Capecitabine is released after 12 hour.

Method of dissolution study is well known in the art. Preferably dissolution study can be carried out in type II or type I dissolution apparatus USP, using suitable buffer or purified water as dissolution medium at 37° C. and 50/75/100 RPM.

In another embodiment, the present invention provides extended release capsules comprising multiple units of Capecitabine, wherein dissolution of Capecitabine from the said composition is extended up to 12 hours after administration, wherein the composition comprises Capecitabine ranging from 150 to 750 mg.

In another embodiment, the present invention provides extended release capsules comprising multiple units of Capecitabine, wherein dissolution of Capecitabine from the said composition is extended up to 12 hours after administration, wherein the composition comprises crystalline or amorphous form of Capecitabine.

In another embodiment, the present invention provides extended release capsules comprising multiple units of Capecitabine, wherein the release is controlled by modified release matrix material.

According to present invention modified release matrix material may include hydrophilic matrix material or hydrophobic matrix material or mixture thereof.

According to present invention modified release matrix material may include, but not limited to hydroxypropyl methylcellulose (HPMC) with different viscosity grade; for example, HPMC K4M, HPMC K100M, HPMC K100LV, hydroxypropylcellulose, hydroxymethylcellulose, hydroxyethylcellulose, hydroxypropyl ethylcellulose, methylcellulose, ethylcellulose, carboxyethylcellulose, carboxymethyl hydroxyethylcellulose, carbomer, sodium carboxymethylcellulose, polyvinylpyrrolidone, hydrophobic polymers, waxes, fats, long-chained fatty acids, fatty alcohols or corresponding esters or ethers or their mixtures, and the like, or mixtures thereof.

In another embodiment, the present invention provides extended release capsules comprising multiple units of Capecitabine, wherein the release is controlled by modified release coating material, wherein modified release coating is applied onto multiple units of Capecitabine.

According to present invention modified release coating material may include pH-dependent coating material or pH-independent coating material or mixture thereof.

According to present invention modified release coating material may include, but not limited to alkyl celluloses such as, e.g. ethyl cellulose, or cellulose esters, such as, e.g. cellulose acetate, cellulose acetate phthalate, hydroxylpropyl methyl cellulose phthalate, poly vinyl acetate phthalate, polymethacrylate, copolymer of ethyl acrylate and methyl methacrylate, ammonio methacrylate copolymers, polyacrylic acid and polyacrylate and methacrylate copolymers, shellac, hydroxyethyl cellulose, hydroxypropyl cellulose, hydroxypropyl methylcellulose, polyvinyl pyrrolidone, and the like, or mixtures thereof.

In another embodiment, the present invention provides extended release capsules comprising multiple units of Capecitabine, wherein the release is controlled by modified release coating on sphere/pellet or modified release coating on multi-particulates.

### EXAMPLES

The present invention has been described by way of example only, and it is to be recognized that modifications thereto falling within the scope and spirit of appended claims, and which would be obvious to a person skilled in the art based upon the disclosure herein, are also considered to be within the scope of this invention.

### Example 1

Extended release capsule comprising mini tablets of Capecitabine wherein release controlled by modified release hydrophilic matrix material

| Sr. No. | Ingredients | % w/w |
|---|---|---|
| 1 | Capecitabine | 50 to 80% |
| 2 | Microcrystalline cellulose | 3% to 5% |
| 3 | Hydroxypropyl Methyl cellulose | 5% to 15% |
| 4 | Hydroxypropyl Methyl cellulose 6 cps | 2% to 4% |
| 5 | Purified water | q.s. |
| 6 | Hydroxypropyl Methyl cellulose | 5% to 15% |
| 7 | Talc | 0.5% to 1.5% |
| 8 | Magnesium stearate | 0.5% to 1.5% |
| 9 | Film coat | 2% to 4% |
| 10 | Empty hard gelatin capsule | 1 No |

Process:
1. Capecitabine, microcrystalline cellulose, hydroxypropyl methyl cellulose were sifted through appropriate sieve.
2. Materials of step 1 were placed in granulator and dry mixed properly.
3. Hydroxypropyl methyl cellulose 6 cps was dissolved in purified water and used to granulate the materials of step 2.
4. Wet mass was dried in dryer.
5. Dried granules were passed through appropriate screen.
6. Hydroxypropyl methyl cellulose and Talc were sifted through appropriate sieve and mixed properly with sized granules.
7. Magnesium stearate was sifted through appropriate sieve and mixed properly with materials of step 6.
8. Lubricated blend was compressed using appropriate tooling to produce mini tablets.
9. Optionally, Film coat was applied on compressed tablets of step 8, prepared from Hydroxypropyl Methyl cellulose, Talc, Titanium, Polyethylene glycol and Purified water.
10. Appropriate number of tablets was filled in empty hard gelatin capsule.

### Example 2

Extended release capsule comprising mini tablets of Capecitabine wherein release controlled by modified release hydrophilic matrix material.

Extended release capsule comprising mini tablets of Capecitabine were prepared as follow: part A: preparation of mini tablet followed by part B: preparation of extended release capsule comprising mini tablets.

A) Preparation of Mini Tablet

| Sr. No. | Ingredients | Mg/mini tablet |
|---|---|---|
| 1 | Capecitabine | 50 |
| 2 | Microcrystalline cellulose | 2.6 |
| 3 | Hydroxypropyl Methyl cellulose | 7 |
| 4 | Hydroxypropyl Methyl cellulose 6 cps | 2 |
| 5 | Purified water | q.s. |
| 6 | Hydroxypropyl Methyl cellulose | 7 |
| 7 | Talc | 0.7 |
| 8 | Magnesium stearate | 0.7 |
| 9 | Film coat | 2 |

Process:
1. Capecitabine, microcrystalline cellulose, hydroxypropyl methyl cellulose were sifted through appropriate sieve.
2. Materials of step 1 were placed in granulator and dry mixed properly.
3. Hydroxypropyl methyl cellulose 6 cps was dissolved in purified water and used to granulate the materials of step 2.
4. Wet mass was dried in dryer.
5. Dried granules were passed through appropriate screen.
6. Hydroxypropyl methyl cellulose and Talc were sifted through appropriate sieve and mixed properly with sized granules.
7. Magnesium stearate was sifted through appropriate sieve and mixed properly with materials of step 6.
8. Lubricated blend was compressed using appropriate tooling to produce tablets at average weight 70 mg.
9. Optionally, Film coat was applied on compressed tablets of step 8, prepared from hydroxypropyl methyl cellulose, Talc, Titanium, polyethylene glycol and purified water.

B) Preparation of Extended Release Capsule Comprising Mini Tablets
1. 10 mini tablets obtained into example 2A were filled in empty hard gelatin capsule.

As per dose requirement, the mini tablets obtained into example 2A were filled into appropriate hard gelatin capsule to prepare extended release capsule comprising mini tablets of Capecitabine. For example, 3 mini tablets can be filled to for 150 mg dose and 10 mini tablets were filled for 500 mg dose.

### Example 3

Extended release capsule comprising mini tablets of Capecitabine wherein release controlled by modified release hydrophilic matrix material.

A) Preparation of Mini Tablet

| Sr. No. | Ingredients | Mg/mini tablet |
|---|---|---|
| 1 | Capecitabine | 50 |
| 2 | Lactose Anhydrous | 2.5 |
| 2 | Microcrystalline cellulose | 3.5 |
| 4 | Hydroxypropyl Methyl cellulose 6 cps | 2.5 |
| 5 | Purified water | q.s. |
| 6 | Hydroxypropyl Methyl cellulose K4 M CR | 7.5 |
| 8 | Magnesium stearate | 1 |

Process:
1. Capecitabine, lactose anhydrous, microcrystalline cellulose, one part of hydroxypropyl methyl cellulose 6 cps were sifted through appropriate sieve.
2. Materials of step 1 were placed in granulator and dry mixed properly.
3. Another part of hydroxypropyl methyl cellulose 6 cps was dissolved in purified water and used to granulate the materials of step 2.
4. Wet mass was dried in dryer.
5. Dried granules were passed through appropriate screen.
6. Hydroxypropyl methyl cellulose K4 M CR and magnesium stearate were sifted through appropriate sieve and mixed properly with sized granules.
7. Lubricated blend was compressed using appropriate tooling to produce tablets at average weight 67 mg.

B) Preparation of Extended Release Capsule Comprising Mini Tablets
1. 3 mini tablets obtained into example 3A were filled in empty hard gelatin capsule to obtain capsule of 150 mg extended release capsule of Capecitabine or
2. 10 mini tablets obtained into example 3A were filled in empty hard gelatin capsule to obtain capsule of 500 mg extended release capsule of Capecitabine.

As per dose requirement, the mini tablets obtained into example 3A were filled into appropriate hard gelatin capsule to prepare extended release capsule comprising mini tablets of Capecitabine. For example, 3 mini tablets can be filled to for 150 mg dose and 10 mini tablets were filled for 500 mg dose.

### Example 4

Extended release capsule comprising mini tablets of Capecitabine wherein release is controlled by modified release hydrophobic matrix material.

| Sr. No. | Ingredients | % w/w |
|---|---|---|
| 1 | Capecitabine | 50 to 80% |
| 2 | Microcrystalline cellulose | 3% to 10% |
| 3 | Ethylcellulose | 5% to 15% |
| 4 | Methanol | q.s. |
| 5 | Talc | 0.5% to 1.5% |
| 6 | Magnesium stearate | 0.5% to 1.5% |
| 7 | Film coat | 2% to 4% |
| 8 | Empty hard gelatin capsule | 1 No |

Process:
1. Capecitabine and microcrystalline cellulose were sifted through appropriate sieve.
2. Materials of step 1 were placed in granulator and dry mixed properly.
3. Ethylcellulose was dissolved in ethanol and used to granulate the materials of step 2.
4. Wet mass was dried in dryer.
5. Dried granules were passed through appropriate screen.
6. Talc was sifted through appropriate sieve and mixed properly with sized granules.
7. Magnesium stearate was sifted through appropriate sieve and mixed properly with materials of step 6.
8. Lubricated blend was compressed using appropriate tooling to produce mini tablets.
9. Optionally, Film coat was applied on compressed tablets of step 8, prepared from Hydroxypropyl Methyl cellulose, Talc, Titanium, Polyethylene glycol and Purified water.
10. Appropriate number of mini tablets was filled in empty hard gelatin capsule.

### Example 5

Extended release capsule comprising spheres comprising Capecitabine prepared by extrusion spheronization technique wherein release is controlled by modified release coating on sphere/pellet.

| Sr. No. | Ingredients | % w/w |
|---|---|---|
| | Core | |
| 1 | Capecitabine | 40 to 70% |
| 2 | Lactose monohydrate | 5% to 10% |
| 3 | Microcrystalline cellulose | 30% to 50% |
| 4 | Hydroxypropyl Methylcellulose E5 | 0.1% to 1% |
| 5 | Purified water | q.s. |
| | Functional coat | |
| 6 | Ethyl cellulose | 3% to 7% |
| 7 | Hydroxypropyl Methylcellulose E6 | 0.1% to 1% |
| 8 | Talc | 0.1% to 1% |
| 9 | Purified water | q.s |
| 10 | Empty hard gelatin capsule | 1 No |

Process:
1. Capecitabine, lactose monohydrate, microcrystalline cellulose were sifted through appropriate sieve.
2. Materials of step 1 were placed in granulator and dry mixed properly.
3. Hydroxypropyl methylcellulose (HPMC E5) was dissolved in Purified water and used to granulate the materials of step 2.
4. Wet mass was subjected to extrusion using extruder.
5. Materials of step 4 were passed through spheronizer to get desired spheres.
6. Wet spheres were transferred to dryer and dried to achieve desired loss on drying.
7. Spheres were subjected to functional coating prepared using Ethyl cellulose, hydroxypropyl methylcellulose E6, talc and purified water.
8. Coated spheres were filled in empty hard gelatin capsule.

### Example 6

Extended release capsule comprising multi-particulates comprising Capecitabine wherein release is controlled by modified release coating on multi-particulates.

| Sr. No. | Ingredients | % w/w |
|---|---|---|
| | Core | |
| 1 | Sugar Spears | 15 to 30% |
| | Drug layer | |
| 2 | Capecitabine | 30 to 75% |
| 2 | Hydroxypropyl Methylcellulose E5 | 1% to 5% |
| 3 | Purified water | q.s. |
| | Functional coat | |
| 6 | Ethyl cellulose | 3% to 7% |
| 7 | Hydroxypropyl Methylcellulose E6 | 0.1% to 1% |
| 8 | Talc | 0.1% to 2% |
| 9 | Purified water | q.s |
| 10 | Empty Hard gelatin capsule | 1 No |

Process:
1. Capecitabine was dissolved in purified water along with hydroxypropyl methylcellulose E5.
2. Drug solution of step was sprayed over sugar spears in fluid bed processor.
3. Drug loaded pellets were subjected to functional coating prepared using ethyl cellulose, hydroxypropyl methylcellulose E6, talc and purified water.
4. Coated pellets were filled in empty hard gelatin capsule.

### Example 7

Extended release capsule comprising multi-particulates comprising Capecitabine wherein release is controlled by modified release coating on multi-particulates.

| Sr. No. | Ingredients | mg/capsule |
|---|---|---|
| | Core | |
| 1 | Sugar Spears | 150.00 |
| | Drug layer | |
| 2 | Capecitabine | 500.00 |
| 2 | Hydroxypropyl Methylcellulose E5 | 10.00 |
| 3 | Purified water | q.s. |
| | Functional coat | |
| 6 | Ethyl cellulose | 30.00 |
| 7 | Hydroxypropyl Methylcellulose E6 | 3.00 |
| 8 | Talc | 7.00 |
| 9 | Purified water | q.s |
| 10 | Empty Hard gelatin capsule | 1 No |

Process:
1. Capecitabine was dissolved in purified water along with hydroxypropyl methylcellulose (HPMC E5).
2. Drug solution of step was sprayed over sugar spears in fluid bed processor.
3. Drug loaded pellets were subjected to functional coating prepared using ethyl cellulose, hydroxypropyl methylcellulose E6, talc and purified water.
4. Coated pellets were filled in empty hard gelatin capsule.

Dissolution Study Results:

The Dissolution study of the extended release capsules comprising multiple units prepared according to examples 2, example 3 and example 7 were carried by in type II dissolution apparatus USP, using 900 ml purified water as dissolution medium at 37° C. and 50 RPM.

Thus, extended release capsules comprising multiple units of Capecitabine suitable for twice daily administration can be prepared according to the present invention wherein dissolution of Capecitabine from the said composition is extended up to 12 hours.

We claim:

1. Extended release capsules comprising a composition of Capecitabine, wherein the dissolution of Capecitabine from the composition is extended up to 12 hours.

2. The extended release capsules according to claim 1, wherein the said composition is in the form of multiple units.

3. The extended release capsules according to claim 2, wherein the multiple units are in form of mini tablet.

4. The extended release capsules according to claim 2, wherein the multiple units are in form of pellet or sphere.

5. The extended release capsules according to claim 2, wherein the multiple units are in form of multi-particulates.

6. The extended release capsules according to claim 2, wherein the dissolution of Capecitabine from the composition is 15 to 35% after 1 hour, 40 to 60% after 4 hour, 60 to 80% after 8 hour and not less than (NLT) 85% of the total amount of Capecitabine released after 12 hour.

7. The extended release capsules according to claim 1, wherein the composition comprises Capecitabine ranging from 150 to 750 mg.

8. The extended release capsules according to claim 2, wherein the release from the multiple units is controlled by modified release matrix material or modified release coating material.

9. The extended release capsules according to claim 8, wherein modified release matrix material comprises hydrophilic matrix material or hydrophobic matrix material or mixture thereof.

10. The extended release capsules according to claim 8, wherein modified release coating material may include pH-dependent coating material or pH-independent coating material or mixture thereof.
